SEQUENCE LISTING (3 sequences)

SEQ ID NO: 1 - protein, 222 aa, Artificial Sequence
  Receptor binding domain of SARS-CoV-2 spike protein (RBD) Arg319 to Phe541 of GenBank accession number YP_009724390.1.
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 1273 aa, Artificial Sequence
  Full length S protein of SARS-CoV-2 , Met1-Thr1261 of GenBank accession number YP_009724390.1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 670 aa, Artificial Sequence
  S1 protein of SARS-CoV-2; Val16-Arg685 of GenBank accession number YP_009724390
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

(19) United States
(12) Patent Application Publication
(10) Pub. No.: US 2023/0054807 A1
(43) Pub. Date: Feb. 23, 2023

(54) NEUTRALIZING MONO-SPECIFIC IgY ANTIBODIES TO INHIBIT OR TREAT SARS-COV-2 CORONAVIRUS INFECTION

(71) Applicant: King Abdulaziz University, Jeddah (SA)

(72) Inventors: Esam Ibraheem AZHAR, Jeddah (SA); Sherif Ali EL-KAFRAWY, Jeddah (SA); Aymn Talat ABBAS, Jeddah (SA)

(21) Appl. No.: 17/387,218

(22) Filed: Jul. 28, 2021

(51) Int. Cl. C07K 16/10; A61P 31/14

(52) U.S. Cl. CPC C07K 16/10; A61P 31/14; C07K 2317/11; C07K 2317/23; C07K 2317/76; A61K 2039/505

(57) ABSTRACT

Methods for producing mono-specific immunoglobin Y (IgY) antibodies against severe acute respiratory syndrome coronavirus 2 (SARS-CoV-2) are disclosed. IgY antibodies are found in birds and can be isolated from egg yolk of chicken eggs. Hens are immunized with a SARS-CoV-2 spike protein comprising an ACE2 receptor binding domain (RBD). IgY antibodies against the RBD are isolated from eggs laid by the hens. The isolated RBD-IgY antibodies were tested in vitro in mammalian cells, wherein the RBD-IgY blocked SARS-CoV-2 infection of the cells. A pharmaceutical composition comprising the mono-specific IgY antibodies can be used to inhibit or treat COOVID-19, the SARS-CoV-2 infection. IgY antibodies are generally regarded as safe and offer various production and treatment advantages when compared to mammalian antibodies.

### SEQUENCE LISTING

This application includes as the Sequence Listing the complete contents of the accompanying text file “Sequence.txt”, created Jul. 6, 2021, containing 19 kilobytes, hereby incorporated by reference.

### BACKGROUND OF THE INVENTION

### Field of the Invention

The invention generally relates methods for producing antibodies against severe acute respiratory syndrome coronavirus 2 (SARS-CoV-2), particularly mono-specific immunoglobin Y (IgY) antibodies isolated from egg yolk. The invention further relates to methods of treating a subject with a pharmaceutical composition comprising the mono-specific IgY antibodies to inhibit or treat SARS-CoV-2 infection.

### Background

According to national and international organizations in charge of public health surveillance, considered that SARS-CoV-2, that is the cause of COVID-19 epidemic is one of the greatest public health problems (Perez de la Lastra, Baca-Gonzalez et al. 2020). It was declared by WHO in 2020 that SARS-CoV-2 is continuously spreading worldwide, with more than 23 million confirmed cases which included more than 800 thousand deaths (Perez de la Lastra, Baca-Gonzalez et al. 2020). The race to develop SARS-CoV-2-specific therapeutics and vaccines is already ongoing (Kupferschmidt and Cohen 2020). The population around the world suffered psychologically and socio-economically because of the absence of specific drugs targeting SARS-CoV-2, leading to a significant number of new confirmed cases and deaths (Li, Ge et al. 2020).

Another possible strategy, rather than vaccine or anti-viral therapeutics that could beat COVID-19, is a passive immunotherapy using neutralizing antibodies (Owji, Negandaripour et al. 2020). The antibody-based immunotherapeutic strategies, such as monoclonal antibodies (MAbs), neutralizing antibodies (NAbs) convalescent plasma and intravenous immunoglobulins (IVIg) could be applied for treating COVID-19 (Sharun, Tiwari et al. 2020). Among the considerations for this approach is that several epitopes should be targeted rather than one epitope for effective passive immunotherapy. Moreover, the long pathway that is needed for monoclonal antibodies to be designed and followed by clinical testing is costly as well as time consuming and labor intensive. These are barriers that are even higher during a pandemic outbreak, which presents an urgent need for effective therapeutics (Owji, Negandaripour et al. 2020). It has been found that better clinical outcomes with higher potency can be achieved when using specific antibodies derived from immunized animals than convalescent plasma therapy. In addition, relative to their plasma counterparts, the possibility of contamination and host reactions would be reduced; dosing and kinetics would also be more consistent and scalable (Owji, Negandaripour et al. 2020). Furthermore, escape-mutants can be avoided due to synergistic neutralization effects that targets more than one epitope. A recent report showed that a mixture of antibodies substantially improved SARS-CoV-2 neutralization as compared to the use of one monoclonal antibody. (Pinto, Park et al. 2020). Alternatively, smaller nanobodies, which are antibodies generated from camelids, and other antibody preparations are being developed as a protective inhalable approach against this novel coronavirus (Konwarh 2020).

Immunoglobulin Y (IgY) is the primary immunoglobulin found in oviparous animals and transferred to the egg yolk. IgY is equivalent to mammalian IgG. Recently, IgY has been given considerate amount of attention as being the potential alternative for passive immunization in order to stop various infectious diseases (Yi, Qin et al. 2018). According to Nguyen, Tumpey et al. (2010); Tsukamoto, Hiroi et al. (2011); Wallach, Webby et al. (2011); Yang, Wen et al. (2014), egg IgY have been successfully used in patients with cystic fibrosis (CF) against respiratory infection with Pseudomonas aeruginosa, influenza virus and bovine respiratory syncytial virus (Ferella, D. Bellido et al. 2012). It has given encouraging results in the treatment of the previously SARS coronavirus, SARS-CoV-1 (Fu, Huang et al. 2006), alongside a wide variety of infections which can be caused by bacteria and virus in human and veterinary medicine (Pereira, van Tilburg et al. 2019). A number of clinical studies have reported for prophylactic as well as therapeutic use of IgY in human medicine as Porphyromonas gingivalis, Streptococcus mutans, Helicobacter pylori, Extended-spectrum beta-lactamases (Phase II) is produced by Klebsiella pneumoniae and E. coli, Clostridium difficile (Phase II), celiac disease, chronic pain and Pseudomonas aeuruginosa (Phase III). The clinical trial was recorded on the databases which was provided by governmental organizations in Europe, Japan and the United States (Leiva, Gallardo et al. 2020).

Additional examples of specific IgY antibodies have provided highly effective treatment and/or prevention of some virus and bacteria causing respiratory diseases, such as influenza A virus (Nguyen, Tumpey et al. 2010, Tsukamoto, Hiroi et al. 2011, Wallach, Webby et al. 2011, Yang, Wen et al. 2014), influenza B virus (Wen, Zhao et al. 2012), SARS coronavirus (Fu, Huang et al. 2006), bovine respiratory syncytial virus (BRSV) (Ferella, D. Bellido et al. 2012) and Mycobacterium tuberculosis (TB) (Sudjarwo, Eraiko et al. 2017). Lung infection caused by Pseudomonas aeruginosa was treated successfully by the use of IgY technology (Kollberg, Carlander et al. 2003). The approval was given by Swedish Medical Products Agency for treating patients with cystic fibrosis (CF) using an anti-Pseudomonas IgY. Moreover, a drug designation for the treatment of CF using IgY Abs in 2008 was approved by the European Medicines Agency (EMEA) (Jahangiri, Owlia et al. 2018).

The low cost of IgY production and use of high technology in the poultry industry permits underdeveloped countries to integrate these technologies very easily in their production process of IgY Abs, providing a key advantage of IgY (Leiva, Gallardo et al. 2020). Furthermore, IgY has not been reported to cause an inflammatory response in the lung, so it can easily and safely be used to inhibit or treat respiratory infections (Kubickova, Majerova et al. 2014, Thomsen, Christophersen et al. 2015). Leiva et al 2020 predicted that the use of egg yolk antibodies will help develop effective novel and safe biologicals for prophylaxis as well as for the treatment of a wide array of health problems which includes infectious diseases which might be affecting the population and other susceptible groups like; immunocompromised, chronic disease patients, children and elder people.

According to (Lu, Zhao et al. 2020, Walls, Park et al. 2020, Zhou, Yang et al. 2020), the SARS-CoV-2 receptor-binding domain (RBD) spike protein binds to the cell receptor, angiotensin-converting enzyme 2 (ACE2) and enables the virus to enter the cell. This clearly suggests that SARS-CoV-2 would not be able to enter the cell if the RBD-ACE2 interaction was blocked. There exists a similarity between the binding mechanism of SARS-CoV RBDs with ACE2 and SARS-CoV-2 (Kirchdoerfer, Wang et al. 2018, Lan, Ge et al. 2020, Wrapp, Wang et al. 2020). RBD-based vaccines are able to induce robust polyclonal antibody responses that work against MERS-CoV and SARS-CoV and can prevent virus from entering a cell (Du, He et al. 2009, Xu, Jia et al. 2019). These results suggests that anti-RBD antibodies should be able to block the entry of SARS-CoV-2 in an effective manner (Ju, Zhang et al. 2020). Lu et al. (J Immun Res. (2020) teaches IgY antibodies that were produced by immunizing hens with various domains from SARS-CoV-2 spike proteins, with 5 domains identified. The specific epitope SIIAYTMSL partially overlaps the S1/S2 cleavage region in SARS-CoV-2 S and is located on the surface of S trimer in 3D structure, close to the S1/S2 cleavage site. Lu teaches that antibody binding at this location physically blocks the access of proteolytic enzymes to S1/S2 cleavage site and impedes S1/S2 proteolytic cleavage, which is crucial to subsequent virus-cell membrane fusion and viral cell entry. Lu contemplates use of IgY antibodies as a neutralizing antibody therapy for SARS-CoV-2 infection. Perez de Lasta et al. (Vaccines 2020, 8, 486; doi:10.3390/vaccines8030486) provides a review of the state of the art of IgY antibodies and possible uses against SARS-CoV-2 infection, proposing their use as both therapeutic and prophylactic. Perez de Lasta describes methods used to produce IgY antibodies against various other viruses and teaches the methodology of producing and isolating IgY antibodies against regions of the spike protein that interact with ACE2 receptors and describes uses as a pharmaceutical composition to prevent or treat SARS-CoV-2. Somasanduram et al. (Internat Immunopharmacol. 85 (2020) 106654) teaches methods for producing monoclonal IgY antibodies against SARS-CoV-2 using a phage display system. Wei et al. (bioRxiv preprint doi:https://doi.org/10.1101/2021.02.16.430255) teaches SARS-CoV-2 S-RBD as an antigen to immunize laying hens in order to extract, separate and purify SARS-CoV-2-IgY from egg yolk. Wei further teaches that SARS-CoV-2-IgY(S-IgY) can block the entry of SARS-CoV-2 into mammalian cells and reduce the viral load in the cells. S-IgY can inhibit the entry and replication of SARS-CoV-2, which is related to its targeting the ACE2 binding domain. Wei teaches that S-IgY is safe, efficient, stable, and easy to obtain and concludes that it may be an effective method for the prevention and treatment of COVID-19 pneumonia.

Despite the recent and rapid advancements in the field, a need exists for additional highly specific antibodies for the treatment and/or prevention of COVID-19. Furthermore, there is a need for production of anti-SARS-CoV-2 antibodies at an industrial scale that is adaptable to a wide variety of geographical locations that is economically feasible to provide a world-wide resource to combat the spread of infection and the resulting morbidity and mortality of COVID-19.

### SUMMARY OF THE INVENTION

The invention relates to mono-specific IgY antibodies against SARS-CoV-2 for administration as a therapeutic agent to inhibit or treat SARS-CoV-2 infection, commonly known as COVID-19. The antibodies are directed against the receptor binding domain of the SARS-CoV-2 spike protein that interacts with the angiotensin-converting enzyme 2 (ACE2) receptor on the surface of a susceptible cell.

One embodiment of the invention is a method of producing immunoglobulin Y (IgY) antibodies against severe acute respiratory syndrome coronavirus 2 (SARS-CoV-2) by immunizing one or more chickens with a quantity of specific epitope sufficient to generate anti-RBD IgY antibodies. The specific epitope encodes a peptide fragment of the receptor binding domain (RBD) of the SARS-CoV-2 spike protein, having the amino acid identity of SEQ ID NO:1. The anti-RBD IgY antibodies are isolated from any number of eggs laid by the one or more chickens that were immunized with the specific epitope. The method is highly scalable and can be conducted by injecting a plurality of chickens such that the anti-RBD IgY is isolated from a plurality of eggs.

When isolated from egg yolk and purified, the anti-RBD IgY is at least 2% of total IgY. In some embodiments, the anti-RBD IgY is in the range of approximately 2-10% of total IgY. In yet other embodiments, the anti-RBD IgY is greater than 10% of total IgY. The anti-RBD IgY antibodies are isolated, purified and formulated in a pharmaceutically acceptable carrier, with the anti-RBD IgY having a titer of at least 1×10⁴. In another embodiment, the antibody titer of anti-RBD IgY is at least 1×10⁵.

In another embodiment, the invention is a method of inhibiting or treating SARS-CoV-2 infection in a subject in need thereof with IgY antibodies against SARS-CoV-2. The method of treatment provides a pharmaceutical composition comprising a therapeutically effective amount of anti-RBD IgY antibodies. The anti-RBD IgY is obtained by immunizing at least one chicken with a quantity of a highly specific epitope encoding a peptide fragment of a receptor binding domain (RBD) of the SARS-CoV-2 spike protein having the amino acid identity of SEQ ID NO:1 and isolating the anti-RBD IgY antibodies against SARS-CoV-2 from an egg or eggs laid by the immunized chicken(s), and preparing a pharmaceutical composition comprising the anti-RBD IgY antibodies. A therapeutically effective amount of the pharmaceutical composition is administered to a subject at risk of contracting a SARS-CoV-2 infection, or to a subject suffering from SARS-CoV-2 infection or COVID-19, wherein the therapeutically effective amount is sufficient to inhibit or treat the SARS-CoV-2 infection. In one embodiment, the therapeutically effective amount is in the range of 1 to 100,000 mg. In another embodiment, the therapeutically effective amount is in the range of 100 mg to 10,000 mg. In another embodiment, the range is 900 to 5,000 mg.

In yet another embodiment, the pharmaceutical composition is a cocktail of anti-RBD IgY and at least one additional IgY antibody directed against a full length spike protein or against a domain, region or fragment of SARS-CoV-2 other than the RBD. In one such embodiment, the cocktail comprises a first antibody, the anti-RBD IgY antibody, and a second antibody, which is directed against the full length S protein. In another embodiment, the cocktail comprises a first antibody, which is the anti-RBD IgY antibody, and a second antibody, which is directed against a fragment of the S protein that is not the RBD or comprises the RBD.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1 shows the SDS-PAGE profile of the purified IgY antibodies. Lane A: standard protein ladder to indicate molecular mass in kilodaltons (kDa). Lane B: purified IgY with the heavy chain and light chain indicated as HC and LC, respectively.

FIG. 2 shows the profile of the purified IgY antibody in a representative western blot. Lane A: standard protein ladder to indicate molecular mass in kDa. Lane B: purified IgY with the heavy chain and light chain indicated as HC and LC, respectively.

FIG. 3 shows egg yolk anti-SARS-CoV-2 RBD IgY antibodies response of chickens after immunization with anti-SARS-CoV-2 RBD recombinant protein.

FIGS. 4A and 4B show an analysis using western blotting and SDS-PAGE under reducing condition. These assays confirm specificity of anti-RBD IgY antibody binding to the RBD protein. FIG. 4A shows a representative analysis of the RBD protein showing the RBD at 26 kDa. FIG. 4B is a representative western blot showing that the anti-RBD IgY antibody specifically binds to the RBD protein.

FIG. 5 shows different concentrations of anti-RBD IgY antibodies tested against SARS-CoV-3 on Vero-E6 cells examined to determine cytopathic effects (CPE). The IC100 neutralization of the antibody was determined as the reciprocal of the highest dilution at which no CPE was observed.

FIGS. 6A and 6B show that IgY confers in vivo protection in virus-challenged mice. Intranasal administration of anti-RBD IgY antibodies before SARS-COV-2 infection reduced weight loss in the RBD group, as shown in FIG. 6A. Administration of the anti-RBD IgY antibodies before SARS-COV-2 infection also reduced the viral titer in homogenized lungs from the RBD group compared with control groups. Groups: untreated=no treatment prior to infection; IgY=treatment with non-specific IgY prior to infection; RBD=treatment with anti-RBD IgY antibodies prior to infection.

### DETAILED DESCRIPTION

The following descriptions and examples illustrate some exemplary embodiments of the disclosed invention in detail. Those of the skill in the art will recognize that there are numerous variations and modifications of this invention that are encompassed by its scope. Accordingly, the description of a certain exemplary embodiment should not be deemed to limit the scope of the present invention.

Disclosed herein are methods and compositions comprising mono-specific IgY antibodies against SARS-CoV-2 for administration as a therapeutic agent to inhibit or treat SARS-CoV-2 infection, commonly known as COVID-19. The antibodies are directed against the receptor binding domain of the SARS-CoV-2 spike protein that interacts with the ACE2 receptor on the surface of a susceptible cell.

To produce the antibodies of the invention, one or more hens are immunized with a specific SARS-CoV-2 RBD antigen at one or more time points. In one embodiment, the hens are injected with the antigen at least three times at intervals of approximately 2 weeks or more. In response to the antigen, specific anti-RBD IgY antibodies are produced and deposited in the yolks of eggs laid by the immunized hens. Deposition of the IgY Abs in the eggs persists for at least 12 weeks beyond immunization, with isolated and purified IgY having a high antibody titer of anti-RBD IgY antibodies. The anti-IgY Abs has a neutralizing effect against live SARS-CoV-2 virus in vitro. Viral neutralization takes place by binding of the IgY Abs to SARS-CoV-2 viral particles and preventing the interaction of ligands on the viral surface with the cell receptors, thereby blocking transmission of the virus to host cells. The highly specific anti-RBD IgY antibodies of the invention provide effective inhibition of and/or treatment for SARS-COV-2 infection. The subject to be treated may be human, non-human primate, canine, feline, murine and other rodent species, genetically modified or humanized experimental animal, camelid, bovine, ovine, and other livestock and/or dairy animal, particularly those intended for human consumption.

One embodiment of the invention is a method of producing IgY antibodies against SARS-CoV-2 by immunizing one or more chickens with a quantity of specific epitope sufficient to generate anti-RBD IgY antibodies. The specific epitope encodes a peptide fragment of the receptor binding domain (RBD) of the SARS-CoV-2 spike protein, having the amino acid identity of SEQ ID NO:1. The anti-RBD IgY antibodies are isolated from any number of eggs laid by the one or more chickens that were immunized with the specific epitope. The method is highly scalable and can be conducted by injecting a plurality of chickens such that the anti-RBD IgY is isolated from a plurality of eggs. The method may be applied on an industrial scale and is well-suited for application at a conventional chicken or egg farm.

When isolated from egg yolk and purified, the anti-RBD IgY is at least 2% of total IgY harvested from the yolk or yolks. The anti-RBD IgY is typically in the range of approximately 2-10% of total IgY but may be present at a concentration greater than 10% of total IgY. The anti-RBD IgY antibodies are isolated, purified and formulated in a pharmaceutically acceptable carrier for administration to a subject at risk of contracting SARS-CoV-2, or to a subject suffering from SARS-CoV-2, which is also known as COVID-19. The anti-RBD IgY in the pharmaceutical composition typically has a titer of at least 1×10⁴. In some embodiments, the antibody titer of anti-RBD IgY is at least 1×10⁵.

As used herein, the terms “SARS-CoV-2 S”, “SARS-CoV-2 S protein” “SARS-CoV-2 spike protein”, “spike protein” and “S protein” are used interchangeably to refer to the spike protein of SARS-CoV-2. The S protein comprises two subunits, S1 and S2. S1 mediates receptor binding and S2 mediates fusion of the virus to the host cell membrane. As used herein, the term “51 protein” is used to refer to the 51 subunit of the S protein but sometimes may also be used interchangeably when referring to the S protein, unless specifically identified otherwise.

As used herein, the terms “anti-RBD”, “anti-RBD IgY”, “anti-RBD IgY Abs”, and “anti-RBD IgY antibodies” are used interchangeably to refer to antibodies against the receptor binding domain of the SARS-CoV-2 spike protein.

As used herein, the terms “antigen” and “immunogen” are used interchangeably. “Antigen” typically designates an entity or epitope that is bound by an antibody and the entity or epitope that induces the production of the antibody. More current usage limits the meaning of antigen to that entity bound by an antibody, while the word “immunogen” is used for the entity that induces antibody production. Where an entity discussed herein is both immunogenic and antigenic, reference to it as either an immunogen or antigen will typically be made according to its intended utility. The terms “antigen” and “antigenic region” refer to the epitope that is also synonymous with the antigen or immunogen.

The terms “peptide”, “polypeptide” and “protein” may be used interchangeably herein, although a protein is typically a linear sequence of about 100 or more amino acids covalently joined by peptide bonds, a polypeptide is typically a linear sequence of about 55 to about 100 amino acids covalently joined by peptide bonds and a peptide is typically a linear sequence of about 55 or fewer amino acids covalently joined by peptide bonds. However, all three are composed of a linear sequence of amino acids and each may refer to a sequence of any length.

In another embodiment, the invention is a method of inhibiting or treating SARS-CoV-2 infection in a subject in need thereof with IgY antibodies against SARS-CoV-2. The method of treatment provides a pharmaceutical composition comprising a therapeutically effective amount of anti-RBD IgY antibodies. The therapeutically effective amount of the pharmaceutical composition is administered to a subject at risk of contracting a SARS-CoV-2 infection, or to a subject suffering from SARS-CoV-2 infection or COVID-19 in an amount is sufficient to inhibit or treat the SARS-CoV-2 infection.

One advantage of the invention is that variations can be quickly designed and used to change the epitope of the IgY directed against SARS-CoV-2. These and any other IgY antibodies can be combined into a cocktail of antibodies targeting epitopes other than the RBD, or an RBD mutant. In this way, escape-mutants can be combated due to synergistic neutralization effects that targets more than one epitope. A mixture of antibodies substantially improves neutralization of virus as compared to the use of one monoclonal antibody. Alternatively, smaller nanobodies, which are antibodies generated from camelids, and other antibody cocktails comprising the anti-RBD IgY are contemplated. Antibodies that may be generated are not limited to those recognizing only the RBD immunogen and may include but are not limited to the full-length S protein, the 51 protein, [any other protein structures or domains?] and fragments thereof. Thus, in yet another embodiment, the pharmaceutical composition is a cocktail comprising anti-RBD IgY antibodies and at least one other IgY antibody directed against SARS-CoV-2 selected from the group consisting of antibodies directed against a full length S protein and a full length 51 protein.

In one aspect, the invention is based on the concept of passive immunization, i.e. the administration of specific antibodies (immunoglobulins) in order to treat or protect a subject from an infection. Clinicians have used passive immunization to prevent or to treat various infections for over a century for diseases such as rabies, diphtheria, tetanus, hepatitis B, respiratory syncytial virus and botulism. The rapid spread of the SARS-CoV-2 virus pandemic is a life-threatening problem for many people in numerous countries. Passive immunization could be a bridging tool to improve the situation of critically ill patients, especially for high-risk groups, such as the elderly or individuals suffering from cancer or immunosuppressive treatments.

Passive immunization has some advantages over vaccines, which confer active immunization. First, protection from vaccination takes longer and often requires several doses to elicit a protective immune response, whereas passive immunization provides a much quicker protection. Second, passive immunization can provide protection in immunosuppressed individuals, who are often at a high risk of acquiring infection and may not be considered candidates for vaccination.

One of the many advantages of the invention is that it provides a non-invasive and pain-free animal-friendly technique for industrial-scale production of antibodies in animals. Low antigen quantities are needed to get an efficient immune response in chicken. Large-scale production of IgY Abs can be achieved with only one chicken, which will produce approximately 22 grams of IgY in one year, with at least 2-10% of this produced in response to the immunogen and thus being specifically targeted. An added advantage is that IgY Abs do not deposit in the muscle tissues. Thus, the anti-RBD Abs can be used to treat food animals without deposition of the antibodies in the meat, avoiding the possible defilement of protocols in countries that prohibit the use of antibiotics in livestock industry.

The existing infrastructure of chicken farms for large-scale production of eggs make it very easy to adopt the production of the anti-RBD IgY Abs at an industrial scale. Handling and storage of the anti-RBD IgY prior to isolation from yolk is easily managed with long-term storage of eggs for at least one year under refrigeration at 4° C. In cases of new viral outbreaks, anti-RBD IgY Abs can be produced within a short period of time (6 weeks from vaccination of hens) and can be formulated in different formulations to provide immediate administration to groups of individuals, thus reducing and managing exposure in environments that are known reservoirs of transmission, such as schools, hospitals and mass transit systems.

Eggs can be stored in large quantities for global use in outbreaks or surges in infection that occur with pandemic. After isolation from egg yolks, the storage and processing of the purified anti-RBD IgY holds various advantages over many other types of antibody preparations and other types of pharmaceutical agents. Purified anti-RBD IgY can remain extremely stable in hot conditions up to 65° C. in aqueous conditions and retain their antigen-binding activity at pH 4-6 in the presence of pepsin, thus allowing for most types of storage, processing and purification applications.

The safety profile of anti-RBD IgY, as well as IgY in general, is superior to mammalian IgG Abs since IgY do not bind to, activate or interact with human Fc receptors or fix mammalian complement components. Despite this, IgY Abs have better binding avidity to targeted antigens. This reduces the probability of triggering potentially dangerous immune or inflammatory responses in subjects receiving the anti-RBD IgY therapy. IgY antibodies have an established record of use to efficiently to treat a variety of infections in humans. For passive immunotherapy the IgY antibodies used can be given to a wide range of individuals belonging to any age and immunodeficient patients and pregnant women can be included.

Without being bound by theory, the evolutionary distance between mammals and birds gives the anti-RBD IgY the advantage of producing antibodies more easily and successfully against conserved mammalian proteins than producing IgG in mammalian species. The large phylogenetic distance between mammals and birds allows IgY antibodies to recognize certain mammalian epitopes that might not be recognized efficiently if mammalian antibodies were used and can be used as valuable tools against difficult target epitopes. These can include antibodies raised in animals, such as goats, rabbits, camels, rodents or other experimental animal species. It is also an advantage over antibody production systems, such as the monoclonal IgG antibodies that are typically produced in engineered cells in vitro at a benchtop to industrial scale.

The anti-RBD IgY can be naturally produced and avoid complications that might arise from many synthetic drugs or therapeutic agents with off-target effects. Due to the high level of specificity for the target pathogen, SARS-CoV-2, the chances for off-target effects and side-effects are further minimized. Since the pharmaceutical composition comprising anti-RBD IgY is not an antibiotic, host microbial populations are unaffected. The sialic acid high content in IgY increases the drug's half-life as compared to those with low sialic acid. Thus, the anti-RBD IgY therapy is likely to have a long circulating half-life that increases efficacy against infection.

Pharmaceutical compositions comprising the anti-RBD IgY Abs are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. The pharmaceutical compositions of the present invention may be administered as an oral form, wherein various thickeners, flavorings, diluents, emulsifiers, dispersing aids or binders and the like may be added. The composition of the present invention may contain any such additional ingredients so as to provide the composition in a form suitable for administration. The final amount of IgY Abs in the formulations may vary. However, in general, the amount in the formulations will be from about 0.01-99%, weight/volume. The amount of antibodies administered to a subject may range from 100 μg to 100,000 mg.

The methods involve administering a pharmaceutical composition comprising anti-RBD IgY Abs in a pharmacologically acceptable carrier to a mammal. The mammal may be a human, but this need not always be the case, as veterinary applications of this technology are also contemplated. In particular, animals known to be vectors of SARS-CoV-2 are contemplated, such as camels and other camelid species. Other species include but are not limited to companion “pets” such as dogs, cats, etc.; food source, work and recreational animals such as cattle, horses, oxen, sheep, pigs, goats, and the like; or even wild animals that may be found to serve as a reservoir of SARS-CoV-2. The pharmaceutical compositions of the present invention may be administered by any of the many suitable means which are well known to those of skill in the art, including but not limited to by injection, inhalation, orally, intranasally, by ingestion of a food product containing the anti-SARS-CoV-2 S IgY Abs, etc. The mode of administration injection may be subcutaneous, intramuscular, intravenous or intraperitoneal. In addition, the compositions may be administered in conjunction with other treatment modalities such as substances that boost the immune system, various anti-bacterial chemotherapeutic agents, antibiotics, and the like.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene[1]polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other nontoxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The present invention also encompasses antibodies to SARS-CoV-2 mutants, RBD mutants, other antigenic regions of SARS-CoV-2 or SARS-CoV-2 mutants, and any other epitopes that are overlapping or complimentary to the RBD of SARS-CoV-2 or SEQ ID NO:1. Such antibodies may be polyclonal or monoclonal antibodies that are generated in chickens/eggs. One or more of these alternative antibodies may be formulated in a pharmaceutical composition along with the anti-RBD IgY Abs.

Before exemplary embodiments of the present invention are described in greater detail, it is to be understood that this invention is not limited to any particular embodiments described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

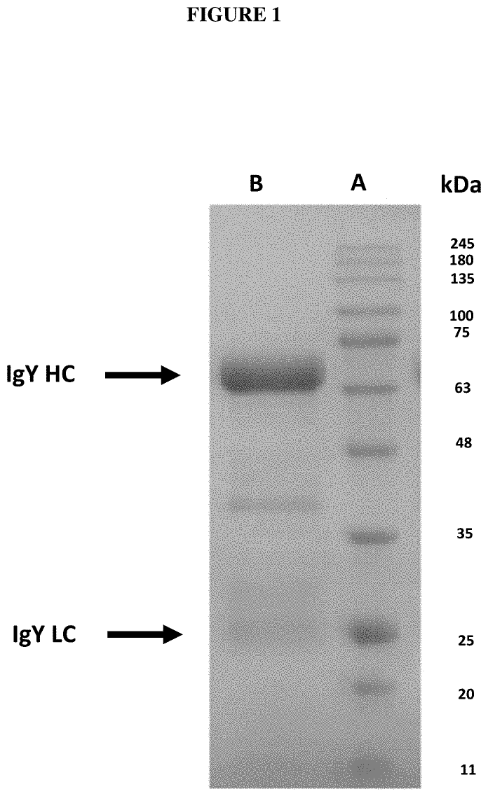

The following Examples provide exemplary designs and methods for fabricating and using microgrippers of the invention. These Examples describe materials and methods for using embodiments illustrated in FIGS. 1-5. Additional details can be found in the section entitled “Brief Description of the Drawings”.

### Materials & Methods

### Immunization of Laying Hens

A total of 8 Lohmann laying hens were purchased from a local retailer (Al-Gharbia Breeding Company), aged 175 days old. These hens were selected solely based on proven egg production. Hens were housed in cages after categorizing them into clusters in dark and light cycle with moderate room temperature (24±3° C.). All the hens were provided with proper food and water.

For the initial immunization on day 0, 200 μg of recombinant SARS-CoV-2 RBD protein was emulsified in overall Freund's Adjuvant at a ratio of 1:1 in complete Freund's adjuvant (Sigma, USA, Cat: F5881) for the first immunization. Complete Freund's Adjuvant, or CFA, is a water in oil emulsion, which also contains inactivated mycobacteria. For the subsequent booster immunizations 200 μg of recombinant SARS-CoV-2 RBD protein was emulsified in partial Freund's Adjuvant for immunization boosters on days 14 and 28. Incomplete Freund's Adjuvant, or IFA, is the same water in oil emulsion, but does not contain the mycobacteria pathogen. A suspension of the recombinant protein to be mixed was taken up through a 19-gauge needle into a 5 ml syringe and agitated with the plunger until the emulsion reached the stability point.

The recombinant SARS-CoV-2 RBD protein immunogen has the amino acid sequence shown in Table 1. In other experiments, the amino acid sequence shown in Tables 2 or 3 can used.

TABLE 1
Amino acid sequence of the receptor binding
domain of SARS-CoV-2 (2019-nCoV) Spike
Protein (RBD), consisting of a fragment
from Arg319 to Phe541

| | |
|---|---|
| GenBank accession number | SEQ ID NO: 1 |
| YP_009724390.1, | |
| Arg319 to Phe541 | |

RVQPTESIVRFPNITNLCPFGEVFNATRFASVYAWNRKRISNCVADYS
VLYNSASFSTFKCYGVSPTKLNDLCFTNVYADSFVIRGDEVRQIAPGQ
TGKIADYNYKLPDDFTGCVIAWNSNNDSKVGGNYNYLYRLFRKSNLKP
FERDISTEIYQAGSTPCNGVEGFNCYFPLQSYGFQPTNGVGYQPYRVV
VLSFELLHAPATVCGPKKSTNLVKNKCVNF

TABLE 2
Amino acid sequence of the full length S
protein of SARS-CoV-2 (2019-nCoV).

| | |
|---|---|
| GenBank accession number | SEQ ID NO: 2 |
| YP_009724390.1, | |
| Met1-Thr1261 | |

MFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRFNGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT

TABLE 3
Amino acid sequence of S1 protein
of SARS-CoV-2 (2019-nCoV).

| | |
|---|---|
| GenBank accession number | SEQ ID NO: 3 |
| YP_009724390; | |
| S1, Val16-Arg685 | |

VNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRV
YSSANNCTFEYVSQPFLMDLEGKQGNFKNLREFVFKNIDGYFKIYSKH
TPINLVRDLPQGFSALEPLVDLPIGINITRFQTLLALHRSYLTPGDSS
SGWTAGAAAYYVGYLQPRTFLLKYNENGTITDAVDCALDPLSETKCTL
KSFTVEKGIYQTSNFRVQPTESIVRFPNITNLCPFGEVFNATRFASVY
AWNRKRISNCVADYSVLYNSASFSTFKCYGVSPTKLNDLCFTNVYADS
FVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSY
GFQPTNGVGYQPYRVVVLSFELLHAPATVCGPKKSTNLVKNKCVNFNF
NGLTGTGVLTESNKKFLPFQQFGRDIADTTDAVRDPQTLEILDITPCS
FGGVSVITPGTNTSNQVAVLYQDVNCTEVPVAIHADQLTPTWRVYSTG
SNVFQTRAGCLIGAEHVNNSYECDIPIGAGICASYQTQTNSPRRAR

Immunization was administered by injection to the selected hens (n=4) into the pectoral muscles on days 0, 14 and 28, with half of the volume injected into the right pectoral muscle and the other half injected in the left pectoral muscle. The control group (n=4) received phosphate buffered saline (PBS) and adjuvant on experimental days 0, 14 and 28. Blood samples were drawn from each bird prior to day 0 injection to establish a baseline that was used to determine the antibody response. Eggs were collected daily beginning one week prior to immunization and then for 12 weeks after the first immunization, beginning at 24 hours post-day 0 injection (i.e., on experimental day 1). Eggs were stored at 4° C. for up to one week until IgY was isolated. The experiment was ended after 12 weeks, with a second blood sample drawn from each bird on the day prior to slaughter. The Unit of Biomedical Ethics Research Committee, Faculty of Medicine, King Abdulaziz University (Permit No: 120-18) reviewed and approved this experimental protocol.

### Isolation and Purification of Yolk IgY

Yolks from all eggs laid by an immunized or control hen were removed and pooled on weekly basis. The yolks were washed with de-ionized water and Pierce Chicken IgY purification kit (Thermo Fisher Scientific, USA) was used for purifying the IgY antibodies from the pooled yolks. This was carried out according to manufacturer's instructions. The concentration of IgY for each weekly pooled sample was calculated using a NanoDrop 2000 spectrophotometer system (Thermo Scientific, USA).

### Sodium Dodecyl Sulfate-Polyacrylamide Gel Electrophoresis

To determine the molecular weight and purity of the isolated IgY, sodium dodecyl sulfate-polyacrylamide gel electrophoresis (SDS-PAGE) was conducted. The assay was carried out in reduced conditions. Protein samples of IgY in its purified form were prepared by mixing with 2× of the sample buffer and boiling for about 10 minutes at a temperature of 100° Celsius. An aliquot of 25 μL protein per well was loaded into a 12% polyacrylamide gel. A pre-stained protein standard marker (MOLEQULE ON-, New Zealand) was loaded into a control lane for comparison with the IgY samples to determine molecular weight. Samples were electrophoresed in a Mini-PROTEAN® 3 cell (Bio-Rad Laboratories, USA) at room temperature in a running buffer (Tris-glycine buffer) at 200 V for a time of 40 minutes. Coomassie Brilliant Blue Stain was used to visualize the protein bands, which were analyzed using GeneTools image analysis software (Syngene, UK).

### Reactivity of Anti-RBD IgY Antibodies

The reactivity and titer of the anti-RBD IgY antibodies was determined by enzyme-linked immunosorbent assay (ELISA). Microtiter plates were coated with 500 ng/ml purified SARS-CoV-2 RBD antigen (Sino Biological, Inc, China) in PBS (0.01M, pH 7.4), at 100 μL/well and stored at 4° C. overnight. The plates were washed with wash buffer three times (lx PBS, tween-20) and non-specific sites were blocked with 250 μl of blocking buffer (5% skim milk in PBS-Tween) at room temperature for one hour, followed by cleaning it three times with the washing buffer. The IgY antibody titers were determined by serially diluting the serum samples obtained from the immunized and control (nonimmunized) hens and a purified IgY. After loading with samples, plates were incubated at 37° C. for 1 h. A 1:10,000 dilution of horseradish peroxidase (HRP)-conjugated rabbit anti-chicken IgY (Abcam, UK) was added to each well (100 μl/well) and incubated for 1 h at 37° C. The plates were washed and the color was developed by adding 100 μl/well TMB substrate solution (Promega, USA) and incubated for half hour. The reaction was then stopped by addition of 100 μl 2M H2SO4 to each well. A microtiter plate reader (ELX800 Biokit) was used to read the optical densities (OD) at 450 nm. PBS was used as a blank control and a purified form of IgY from non-immunized hens served as a negative control. Anti-RBD IgY titer was assessed as the maximum dilution of the sample which showed an OD value 2.1 times the reading of the negative control.

### Western Blot Assay

To determine the specificity of the anti-SARS-CoV-2 RBD IgY antibodies, the western blotting technique was applied in accordance with the previous method mentioned but with certain modifications [49]. An amount of 500 ng recombinant RBD protein was mixed with 20 μl electrophoresis sample buffer and subjected to SDS-PAGE in a 14% slab polyacrylamide gel separated by a 4% stacking gel at 200 V for 40 minutes at room temperature. The gel and the blotting papers were equilibrated in transfer buffer for 10 minutes. After equilibration, the RBD protein sample(s) were electrically mobilized and transferred onto Polyvinylidene fluoride (PVDF) membrane which was activated by methanol (Thermo Fisher, USA) at 30V overnight. The PVDF membrane was cut into strips measuring 0.5-cm and blocked with Tris-buffered saline with 0.1% Tween 20 (TBS-T) and 5% non-fat dry milk for an hour at room temperature. The PVDF strips were then washed three times for ten minutes, followed by incubation in a 1:50 dilution of anti-SARS-CoV-2 RBD IgY antibodies. Post-incubation, the strips were cleaned three times with TBS-T for ten minutes and incubated with HRP-conjugated rabbit anti-chicken IgY H&L (having both the heavy and light chains present) (Abcam, UK) at 1:10,000 dilution in blocking buffer for one hour at room temperature. Then the strips were washed again 3 times for ten mins. After washing, the strips were incubated with HRP colorimetric substrate (Immun-Blot Opti-4 CN colorimetric Kit, Bio-Rad) for fifteen minutes at room temperature. The reaction was stopped by washing the strips with distilled water. After visible bands developed, the strips were photographed.

### Neutralization Assay

All neutralization assays were conducted at the Special Infectious Agents Unit at King Fahd Medical Research Center (KFMRC), King Abdul-Aziz University, Jeddah, which contains live viruses in a suitable biosafety level facility. These experiments followed the suggested safety measures and precautions. A method by Iwata-Yoshikawa, Okamura et al. (2019) was used to complete the neutralizing assay. To be precise, SARS-CoV-2 isolates were added with an approximate volume of serial dilutions of the IgY antibodies for 1 hour based on the absence and occurrence of IgY antibodies. Afterward, this mixture, in the presence of viral inoculation medium, was injected onto Vero E6 cells in triplicates in 98 wells plates. The humidification of incubated cells was 5% CO2 at 37° C. for two or three days in positive virus control wells until achieving 80-90% cytopathic effect (CPE). The neutralizing antibody titers were determined as reciprocal of the highest dilution at which no CPE were observed.

### Animals

Specific pathogen-free 8-10-week-old female C57BL/6 mice were purchased from Charles River Laboratories and maintained in the Animal Care Facilities University of Iowa. All protocols were approved by the Institutional Animal Care and Use Committees of the University of Iowa (Animal Approval Number: 9051795). The SARS-CoV-2 strains used in this research were isolated from COVID-19 patients and passaged on Vero E6 and Calu-3 2B4 cells.

### Mouse Infection Protocol

8-10-week-old female C57BL/6 mice were anesthetized with ketamine and transduced intranasally with 2.5×10⁸ PFU of Ad5-ACE2. Five days post transduction, mice were intranasally administered 0.25 mg of the IgY-Ab (anti-RBD IgY or adjuvant IgY as control) then 2 h later, the mice were infected intranasally with SARS-CoV-2 (1×10⁵ PFU) in a total volume of 50 μL DMEM.

### Viral Titer in the Mouse Lungs

One group of animals was sacrificed after 2 days and another group after 6 days post-infection (p.i.) (n=4). Lungs were removed, placed into PBS and homogenized using a manual homogenizer. Virus was titrated on Vero E6 cells. Cells were fixed with 4% formaldehyde and plaques were visualized by staining with 0.1% crystal violet.

### Example 1

### Isolation and Purification of IgY

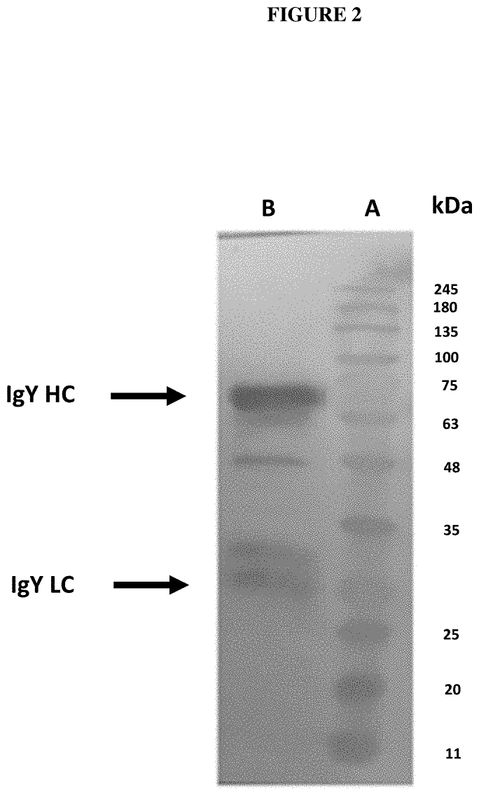

Total IgY was isolated from eggs laid by immunized hens. Representative images of SDS-PAGE, shown in FIG. 1, and western blotting, shown in FIG. 2, demonstrate that the preparation of IgY disassociated into two band of proteins, a major band at ˜68 kDa (heavy chain) and a minor band at ˜27 kDa (light chain) with a 90% purity. Each egg yolk had an average volume of 15 ml, and approximately 75 mg of total IgY was isolated from a single egg. Thus, the total IgY concentration was 5 mg/ml of egg yolk on average.

### Example 2

Anti-RBD IgY Titer in Serum and Total IgY Isolated from Egg Yolk
The amount of anti-RBD IgY in chicken serum and in the total IgY isolated from egg yolk was measured by determining the anti-RBD titer.

Serum collected from hens after the first immunization showed a fixed increase in SARS-CoV-2 RBD specific IgY titers that reached a peak at 7 weeks and remained high until the 12^{th} week (data not shown). In contrast, serum from the control hens injected with PBS-adjuvant only showed no SARS-CoV-2 RBD reaction (data not shown).

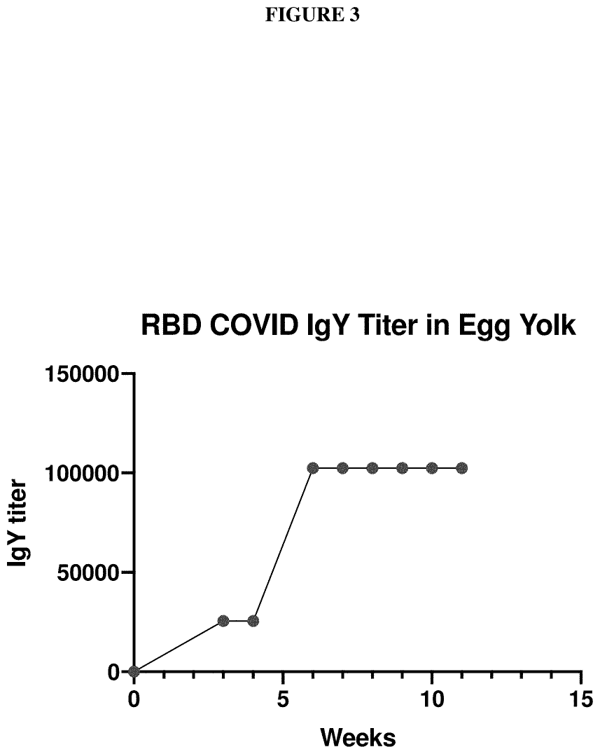

Anti-RBD IgY Abs titers were also measured in weekly pooled samples of egg yolks. Low levels of anti-SARS-CoV-2 RBD IgY antibodies were detected in the eggs at week 3 after the immunization, at a titer of approximately 1×10⁴, as shown in FIG. 3. The titers in eggs from immunized hens reached a peak of approximately 1×10⁵ at the 7^{th} week and maintained this level until the 12^{th} week when the experiments were ended.

### Example 3

### Immunoreactivity of Anti-RBD IgY Against SARS-CoV-2

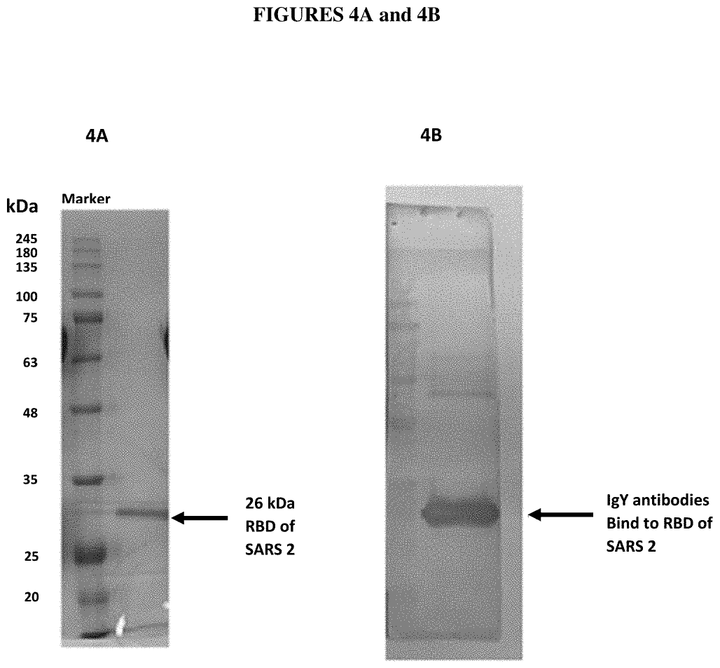

Immunoreactivity of the anti-RBD IgY Abs isolated from egg yolks was assayed. FIG. 4A is a western blot with the arrow indicating the 26 kDa RBD protein. The IgY Abs induced by SARS-COV2-RBD recognized the 47 kDa RBD recombinant protein when the blot was probed with anti-RBD IgY. FIG. 4B shows a representative SDS-PAGE immunoblot that confirmed specificity of anti-RBD IgY antibody binding to the RBD protein.

### Example 4

### Anti-RBD IgY Neutralizes SARS-CoV2 and Inhibits Infection of Mammalian Cells

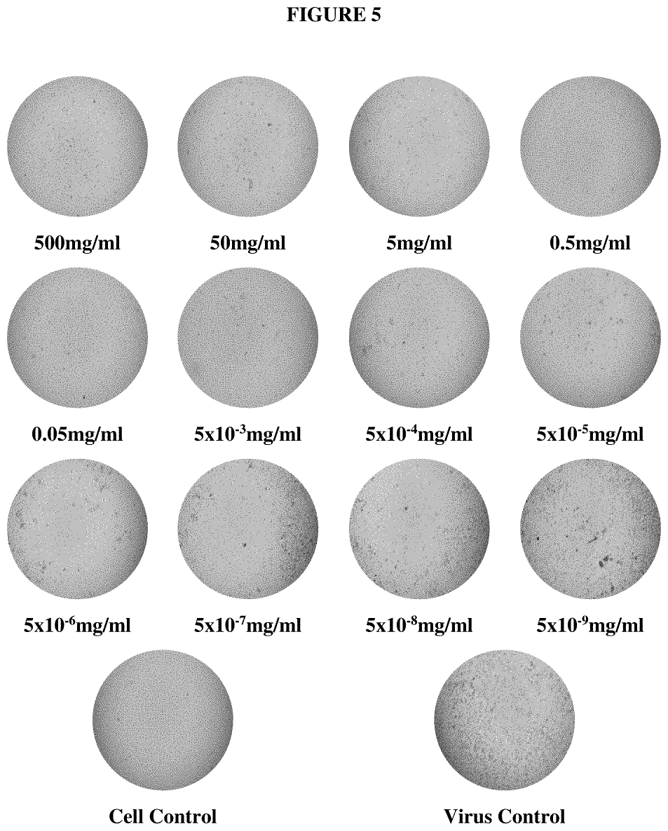

Infectivity of SARS-CoV-2 was tested in vitro. The anti-RBD IgY strongly neutralized infection in permissive Vero cells incubated with live SARS-CoV-2, having an ND₁₀₀ at a dilution of <0.05 μg/ml, as shown in FIG. 5. In contrast, the IgY isolated from adjuvant-injected control chickens demonstrated no antiviral activity against SARS-CoV-2 infection up to 1 mg/ml. These data demonstrate that immunization of chickens with SARS-CoV-2 RBD produced anti-RBD IgY antibodies with a potent ability to inhibit SAR-COV-2 infection.

### Example 5

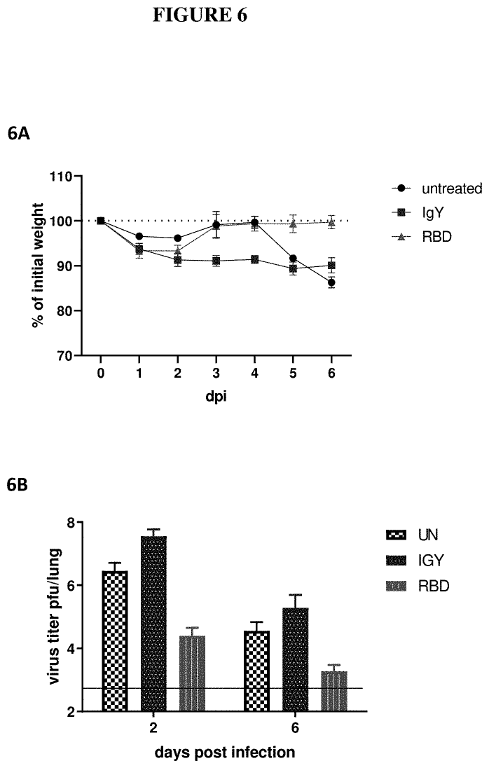

Purified anti-RBD IgY antibodies protect mice from SARS-COV-2 infection. Mice in the test group received intranasal administration of the anti-RBD IgY antibodies, followed by SARS-COV-2 infection (RBD group). Prior to infection with SARS-COV-2, the control groups received a non-specific IgY (IgY group) or no treatment (untreated group). The mice in all groups initially lost weight. However, mice in the RBD group that received anti-RBD IgY prophylactic treatment recovered quickly and maintained their weight regain as illustrated in FIG. 6A. The anti-RBD IgY prophylactic treatment group, at both day 2 and day 6 post infection was protected from viral reproduction compared with control groups, as shown in FIG. 6B.

### Example 6

A mixture of purified anti RBD IgY antibodies can be mixed with purified antibodies raised against the full length S protein (shown in Table 2) and/or the 51 protein (shown in Table 3). A cocktail containing the anti-RBD IgY and one or more of the anti-S and anti-S1 antibodies can provide an enhanced treatment for or protection against SARS-COV-2.

### REFERENCES

Du et al. (2009). Nature Reviews Microbiology 7(3): 226-236.
Ferella et al. (2012). Procedia in Vaccinology 6(Supplement C): 33-38.
Fu et al. (2006). J Virol Methods 133(1): 112-115.
Jahangiri et al. (2018). J Appl Microbiol. 2019 February; 126(2):624-632
Ju et al. (2020). Nature 584(7819): 115-119.
Kirchdoerfer et al. (2018). Scientific reports 8(1): 1-11.
Kollberg et al. (2003). Pediatr Pulmonol 35(6): 433-440.
Konwarh, R. (2020). Frontiers in Immunology 11.
Kubickova et al. (2014). Neuro Endocrinol Lett 35(Suppl 2): 99-104.
Kupferschmidt & Cohen (2020). Race to find COVID-19 treatments accelerates, American Association for the Advancement of Science.
Lan et al. (2020). Nature 581(7807): 215-220.
Leiva et al. (2020). International Immunopharmacology 81: 106269.
Li et al. (2020). Brain, behavior, and immunity. 2020 August; 88:916-919
Lu et al. (2020). The Lancet 395(10224): 565-574.
Nguyen et al. (2010). PLoS One 5(4): e10152.
Owji et al. (2020). International Immunopharmacology: 106924.
Pereira et al. (2019). International Immunopharmacology 73: 293-303.
Perez de la Lastra et al. (2020). Vaccines 8(3): 486.
Pinto et al. (2020). Nature: 2020 July; 583(7815):290-295
Sharun et al. (2020). Expert Opinion on Biological Therapy 20(9): 1033-1046.
Sudjarwo et al. (2017). J Adv Pharm Technol Res 8(3): 91-96.
Thomsen et al. (2015). Infection and Immunity 83(7): 2686-2693.
Tsukamoto et al. (2011). Mol Med Rep 4(2): 209-214.
Wallach, et al. (2011). Clinical and Vaccine Immunology 18(7): 1083-1090.
Walls et al. (2020). Cell. December 10; 183(6):1735
Wen et al. (2012). Antiviral Research 93(1): 154-159.
Wrapp et al. (2020). Science 367(6483): 1260-1263.
Xu et al. (2019). Emerging microbes & infections 8(1): 841-856.
Yang et al. (2014). Journal of Virological Methods 206: 19-26.
Yi et al. (2018). Fish Shellfish Immunol 80: 534-539.
Zhou et al. (2020). Nature 579(7798): 270-273.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1-7. (canceled)

8. A method of inhibiting or treating severe acute respiratory syndrome coronavirus 2 (SARS-CoV-2) infection in a subject in need thereof, comprising the steps of
a) immunizing at least one chicken with at least 200 μg peptide fragment of a receptor binding domain (RBD) of the SARS-CoV-2 spike protein consisting of the amino acid sequence of SEQ ID NO:1 on days 0, 14 and 28; and immunizing at least one additional chicken with at least 200 ug peptide consisting of at least one amino acid sequence selected from the group consisting of SEQ ID NO:2 and SEQ ID NO:3 on days 0, 14 and 28;
b) isolating a first preparation of total IgY antibodies from a yolk of at least one egg laid by the at least one chicken immunized with the peptide fragment consisting of SEQ ID NO: 1₁ and isolating at least one additional preparation of total IgY antibodies from a yolk of at least one egg laid by the at least one additional chicken;
c) combining the first preparation and at least one additional preparation of total IgY antibodies;
d) preparing a pharmaceutical composition comprising a cocktail of the first preparation and the at least one additional preparation of the total IgY antibodies in a pharmaceutically acceptable carrier; and
e) administering a therapeutically effective amount of the pharmaceutical composition to the subject, wherein the therapeutically effective amount is sufficient to inhibit or treat the SARS-CoV-2 infection.

9-13. (canceled)

14. The method of claim 8, wherein the route of administration in step d) of the pharmaceutically acceptable composition is by intravenous injection or infusion, intraperitoneal injection, intraperitoneal infusion, intranasal or oral.

15. The method of claim 8, wherein the route of administration in step d) of the pharmaceutically acceptable composition is by intravenous injection or infusion.

16. A method of inhibiting or treating severe acute respiratory syndrome coronavirus 2 (SARS-CoV-2) infection with a pharmaceutical composition comprising a mixture of total IgY in a subject in need thereof, comprising the steps of
a) immunizing at least one chicken at least three times at intervals of two weeks with at least 200 μg of a peptide fragment of a receptor binding domain (RBD) of a SARS-CoV-2 spike protein consisting of the amino acid sequence of SEQ ID NO:1;
b) immunizing at least one chicken at least three times at intervals of two weeks with at least 200 ug peptide consisting of the amino acid sequence of SEQ ID NO:2;
c) immunizing at least one chicken at least three times at intervals of two weeks with at least 200 ug peptide consisting of the amino acid sequence of SEQ ID NO:3;
d) collecting at least one egg laid by the at least one immunized chicken from each of steps a), b) and c);
e) isolating total IgY antibodies from a yolk of each egg collected in step d);
f) preparing a pharmaceutical composition comprising a mixture of the total IgY antibodies isolated in step e) in a pharmaceutically acceptable carrier; and
g) administering a therapeutically effective amount of the pharmaceutical composition to the subject by a route selected from the group consisting of intravenous injection or infusion, intraperitoneal injection, intraperitoneal infusion, subcutaneous injection, intramuscular injection, intranasal and oral administration, wherein the therapeutically effective amount is sufficient to inhibit or treat the SARS-CoV-2 infection.

17. The method of claim 16, wherein the route of administration in step d) is by intravenous injection or infusion.
